(19) United States
(12) Patent Application Publication
(10) Pub. No.: US 2016/0287911 A1
(43) Pub. Date: Oct. 6, 2016

(54) METHOD OF TREATING SKIN CANCER USING LOW INTENSITY ULTRASOUND

(71) Applicant: SONIFY BIOSCIENCES, LLC, Baltimore, MD (US)

(72) Inventor: Miriam Sara BOER, Baltimore, MD (US)

(73) Assignee: SONIFY BIOSCIENCES, LLC, Baltimore, MD (US)

(21) Appl. No.: 15/037,251

(22) PCT Filed: Oct. 31, 2014
(86) PCT No.: PCT/US14/63279

§ 371 Date: May 17, 2016

Related U.S. Application Data
(60) Provisional application No. 61/907,763, filed on Nov. 22, 2013.
(60) Provisional application No. 61/989,836, filed on May 7, 2014.

(51) Int. Cl. A61N 7/02; A61B 5/00

(52) U.S. Cl. CPC A61N 7/02; A61B 5/444; A61B 5/4836; A61N 2007/0034

(57) ABSTRACT

The present disclosure relates to methods of treating skin cancer using low intensity ultrasound. Low intensity ultrasound can selectively kill skin cancer cells. In some embodiments, the low intensity ultrasound can be continuous and focused.

### CROSS-REFERENCE TO RELATED APPLICATION

This application claims benefit under 35 U.S.C. §119(e) of U.S. Provisional Application No. 61/907,763 filed Nov. 22, 2013, and U.S. Provisional Application No. 61/989,836 filed May 7, 2014, the contents of each of which are incorporated herein by reference in their entirety.

### TECHNICAL FIELD

The present disclosure relates generally to methods of treating skin cancer using low intensity ultrasound.

### BACKGROUND

Skin cancer is the most common form of cancer in the United States. The prevalence of skin cancer is evident by the statistics that one in five Americans will develop skin cancer in the course of a lifetime.

The current treatment options for skin cancer include excisional surgery, radiation therapy, chemotherapy and immunotherapies, each of which has its own limitations. Excisional surgery is the most common method to treat skin cancer. However, because it is very difficult to remove all cancer cells, the likelihood of the cancer growing back can be high for certain types of cancers. Radiation therapy can result in adverse side effects such as chronic radiation dermatitis, skin atrophy and telangiectasia. Chemotherapy has adverse side effects as well, and its overall efficacy is low due to skin cancer's external presence. Immunotherapies are also plagued by serious side effects, including damages to other healthy organs, fever and severe tiredness. Accordingly, there is a strong need in the art to develop new methods to treat skin cancer.

### SUMMARY

The inventor has discovered, inter alia, a survival differential of cells exposed to low intensity ultrasound that correlated with cellular metabolic activities. Specifically, after ultrasound exposure, cells with lower metabolic activities tend to survive better than cells with higher metabolic activities. Accordingly, provided herein is a method of disrupting or killing cells with high metabolic activities, the method comprising exposing cells with high metabolic activities to low intensity ultrasound. Without wishing to be bound by theory, skin tumor cells tend to have high metabolic activities, and normal skin cells tend to have low metabolic activities.

A related aspect of the invention concerns a method of treating a tumor on the skin of a subject using low intensity ultrasound, the method comprising exposing an area of the skin containing the tumor to low intensity ultrasound. In some embodiments, the skin tumor is a skin cancer. The ultrasound frequency, intensity, and the duration of exposure are selected to provide maximal therapeutic results. In some embodiments, the ultrasound frequency is in the range of about 27 kHz to about 2.2 MHz. In some embodiment, the ultrasound intensity is in the range of about 0.1 W/cm² to 5 W/cm². In some embodiments, the duration of exposure is no more than 60 minutes per exposure session. In some embodiments, the low intensity ultrasound is continuous. In some embodiments, the low intensity ultrasound is focused.

The method provided herein can further comprise the following steps: (i) imaging the skin cancer; (ii) providing input to a control unit as a function of the imaging, wherein the control unit controls an ultrasound transducer; and (iii) adjusting the ultrasound focus, intensity, spatial distribution, or any combination thereof as a function of the input.

Skin cancers include, for example, basal cell carcinoma, squamous cell carcinoma, and melanoma. In some embodiments, the skin cancer is melanoma.

### BRIEF DESCRIPTION OF THE DRAWINGS

FIG. 1 is a plot of percent survival following treatment of both A375 and HEKa.

FIG. 2 is a plot of the metabolic activity of the untreated HEKa control that was used in FIG. 1 to calculate percent growth.

FIG. 3 is a plot of the metabolic activity of the untreated A375 control that was used in FIG. 1 to calculate percent growth. Note that the maximum amount of metabolic activity is higher than HEKa in FIG. 2.

FIG. 4 shows percent growth following treatment in a second trial. In this trial the HEKa had significantly higher metabolism and was thus affected more strongly by the low intensity focused ultrasound (LIFU) treatment.

FIG. 5 shows the metabolic activity of HEKa in the second trial. Note that the maximum for the metabolic activity of HEKa is quite high, as the HEKa used in this trial came from fresh stock. Cells from this stock also had a different morphology in addition to the higher metabolism.

FIG. 6 shows the metabolic activity of A375 from the second trial. The maximum amount of metabolic activity is also high, consistent with the A375 control plot from the first trial. A375 ages slowly in culture.

FIG. 7 shows survival HEKa from a third trial, showing a high survival rate following ultrasound treatment. HEKa used in this trial was aged and had a lower metabolism. This plot was the product of 4 trials, and each point is the average of 4 sets of 8 data points. The error bars are one full standard deviation.

FIG. 8 shows metabolic activity of HEKa in the third trial. The lower metabolism correlates with higher survival, confirming that the effects of low intensity ultrasound at the particular frequency and intensity correspond to metabolic activity.

FIG. 9 is a plot showing the average optical density of A375 melanoma strain, untreated control and treated cells. The cellular concentration is on the x-axis, and the average optical density (OD), which reflects the metabolic rate, is on the y-axis. Each control data point is n=8, and each treated data point is n=32. The final two data points are absent on the control plot because they exceeded an average OD of 4, which is the limit of the plate reader. The graph shows a statistically significant decrease in the treated cells' growth in the five highest cell concentrations (the five rightmost data points).

FIGS. 10A-10B are plots showing the average optical density of HEKa following ultrasound treatment. FIG. 10A shows experimental data collected from HEKa noncancerous keratinocytes, untreated and treated cells. The cellular concentration is on the x-axis, and the average optical density (OD), which reflects the metabolic rate, is on the y-axis. Each control data point is n=8, and each treated data point is n=24. In this case, there are only two points of statistically significantly decreased metabolic rates in treated cells, implying the healthy cells are less affected by the ultrasound than the cancerous ones. FIG. 10B shows experimental data of HEKa cells, with each plate as a separate curve. The plates are numbered in order of their treatment. HEKa 1 was run first, HEKa 2 run second, etc. As the runs increased, the survival became more erratic. The explanation for this likely lies in the increasing water bath temperature. For the HEKa cells, their survival seems to be temperature dependent.

FIGS. 11A-11B are plots showing the average optical density of HEMa following ultrasound treatment. FIG. 11A shows experimental data collected from HEMa noncancerous melanocytes, untreated control and treated cells. The cellular concentration is on the x-axis, and the average optical density (OD), which reflects the metabolic rate, is on the y-axis. Each control data point is n=8, and each treated data point is n=32. At first glance, it appears that healthy melanocytes are negatively affected by the ultrasound treatment. However, the data shown in FIG. 11B indicate that the negative impact is clearly linked to the temperature of the water bath. FIG. 11B graphs each plate separately, revealing marked temperature dependence. The plates are numbered in order of their treatment. Here, each data point is n=8.

### DETAILED DESCRIPTION

The inventor has discovered, inter alia, a survival differential of cells exposed to low intensity ultrasound that correlated with cellular metabolic activities. Specifically, after ultrasound exposure, cells with lower metabolic activities tend to survive better than cells with higher metabolic activities. Metabolic activities for cells can be measured by assays such as the WST-1 cell proliferation assay. Accordingly, provided herein is a method of disrupting or killing cells with high metabolic activities, the method comprising exposing cells with high metabolic activities to low intensity ultrasound.

Without wishing to be bound by theory, skin tumor cells typically have higher metabolic activity than normal skin cells as evident by the fact that skin tumor cells reproduce at a faster rate. Based on the inventor's discovery, skin cancer cells are more susceptible to low intensity ultrasound than normal skin cells. The term “susceptible” refers to higher percentage of death for skin cancer cells than normal cells when exposed to the same low intensity ultrasound. The percentage of death for skin cancer cells can be at least 10%, 20%, 30%, 40%, 50%, 60%, 70%, 80%, or 90% higher than normal cells. As used herein, the term “low intensity ultrasound” refers to ultrasound having a frequency of 20 kHz or higher and an intensity at the focal zone of no more than 75 W/cm². Accordingly, a related aspect of the invention concerns a method of treating a skin tumor on the skin of a subject using low intensity ultrasound, the method comprising exposing an area of the skin containing the tumor to low intensity ultrasound. In some embodiments, the skin tumor is benign. In some embodiments, the skin tumor is premalignant. In some embodiments, the skin tumor is a skin cancer.

Skin cancers amenable to treatment with the methods of the present invention include, for example, basal cell carcinoma, squamous cell carcinoma, and melanoma. In some embodiments, the skin cancer is melanoma. The skin cancer can have any size or shape. The skin cancer can include at least one cancer cell, at least 100 cancer cells, at least 1000 cancer cells, at least 10⁵ cancer cells, at least 10⁷cancer cells, at least 10⁹ cancer cells, or at least 10¹² cancer cells. The skin cancer can exist at any location of the skin. Methods of diagnosing skin cancer include, but are not limited to, skin biopsy.

The ultrasound used in the present invention can be produced by an ultrasound source. In some embodiments, the ultrasound source is an ultrasound transducer. In some ultrasound transducers, a piezoelectric element can convert an electrical current to ultrasound. In some other ultrasound transducers, magnetostrictive materials can produce ultrasound when exposed to a magnetic field. Technologies of ultrasound transducers are well known in the art and are not discussed in detail here. It is contemplated that any ultrasound transducer that can provide the desirable ultrasound characteristics (e.g., frequency, focus, and/or intensity) can be used for the purpose of this invention.

It should be noted that the ultrasound frequency, intensity, and the duration of exposure are selected to provide maximal therapeutic results, based in part on the type and size of the skin cancer. In some embodiments, the ultrasound frequency, intensity, and the duration of exposure are selected to induce mechanical damages to cancer cells. Mechanical damages can include, but are not limited to, damage to the cancer cell membrane, damage to one or more organelles inside the cancer cell, and damage to the nucleus membrane. In some embodiments, the ultrasound frequency, intensity, and the duration of exposure are selected to induce apoptosis of cancer cells. In some embodiments, the method of treatment provided herein results in partial or complete remission. In some embodiments, the method of treatment provided herein prevents the skin cancer from spreading.

In some embodiments of the invention, the ultrasound is focused. Similar to light, ultrasound can be focused using a variety of techniques. For example, ultrasound can be focused into a focal zone via a lens, a curved transducer, a phased array, or any combination thereof. Classified in another way, ultrasound can be focused geometrically (e.g., using a lens or a curved transducer) or electronically (e.g., using a phased array). As used herein, the term “focal zone” is defined as an area where the ultrasound energy converges. Typically, the focal zone is the area at which the ultrasound beam is at its narrowest and the ultrasound intensity is the greatest. The ultrasound intensity at the focal zone is determined by dividing the ultrasound energy per unit time at the focal zone by the area of the focal zone. In some embodiments, for ultrasound of the same frequency, a user can control the area of the focal zone by varying how tightly the ultrasound is focused. This in turn can change the ultrasound intensity at the focal zone.

When the skin containing cancer is exposed to low intensity ultrasound, at least 5%, 10%, 20%, 30%, 40%, 50%, 60%, 70%, 80%, 90%, or 99% by volume of the skin cancer is within the focal zone of the ultrasound.

In alternative embodiments, the ultrasound is not focused.

In some embodiments, the ultrasound is continuous, meaning that without changing the intensity setting, the ultrasound intensity stays mostly constant over a period of at least 1 minute, at least 5 minutes, at least 10 minutes, at least 20 minutes, at least 40 minutes, or at least 60 minutes. As used herein, the term “mostly constant” means that the intensity value is within ±5% of a desired value.

In some embodiments, the ultrasound is pulsed, meaning that the ultrasound is delivered in pulses, and the ultrasound intensity varies over time. For pulsed ultrasound, the width of each pulse can be no more than 5000 microseconds, 2500 microseconds, 1000 microseconds, 200 microseconds, 100 microseconds, 50 microseconds, or 20 microseconds. The pulse repetition rate can be more than 10 Hz, 50 Hz, 100 Hz, 200 Hz, 500 Hz, or 1000 Hz.

The ultrasound frequency used in this invention can be in the range of 20 kHz to 100 MHz, 20 kHz to 50 MHz, 20 kHz to 25 MHz, 20 kHz to 10 MHz, 20 kHz to 5 MHz, 20 kHz to 2.5 MHz, or 27 kHz to 2.2 MHz. In some embodiments, the ultrasound frequency is about 27 kHz. In some embodiments, the ultrasound frequency is about 2.2 MHz. In some embodiments of the invention, the ultrasound intensity is not 20 kHz or 2 MHz.

Ultrasound of one, two, three or more frequencies can be used to the same subject, and the intensity at each ultrasound frequency can be the same or different. In some embodiments, different ultrasound frequencies are used simultaneously. In some embodiments, different ultrasound frequencies are used sequentially. In some embodiments, different ultrasound frequencies are used at different stages of treatment (e.g., early, middle, or late stages).

The present invention uses ultrasound that is distinct from high intensity focused ultrasound (HIFU), a class of clinical therapies that use hyperthermic processes to treat diseases. Typically, HIFU transducers deliver ultrasound with high intensities, in the range of 100-10,000 W/cm² to the focal zone, to locally heat and destroy unwanted tissues. Therefore, HIFU does not discriminate between unwanted tissues (e.g., a tumor) and healthy tissues. In contrast, the present invention does not depend on heat to kill cancer cells, and it exploits the survival differential between skin cancer cells and normal skin cells when exposed to low intensity ultrasound. The ultrasound intensity at the focal zone used in the present invention is no more than 75 W/cm², no more than 50 W/cm², no more than 25 W/cm², no more than 10 W/cm², no more than 5 W/cm², no more than 2 W/cm², no more than 1 W/cm², or no more than 0.5 W/cm². In some embodiments, the ultrasound intensity at the focal zone is in the range of about 0.1 to 10 W/cm², 0.1 to 5 W/cm², 0.5 to 10 W/cm², 0.5 to 5 W/cm², 1 to 10 W/cm², or 1 to 5 W/cm². The exact intensity used in the treatment should depend on factors such as severity of the disease, patient gender and age, etc., and can be extrapolated from animal studies. Low intensity ultrasound can allow prolonged exposure of the skin to ultrasound compared to HIFU.

Regardless of ultrasound intensity, because skin can absorb ultrasound and convert it to heat, the longer the skin is exposed to ultrasound, the hotter the skin becomes. Factors such as the ultrasound frequency, intensity and duration of exposure contribute to heat generation. As used herein, the term “duration of exposure” refers to the amount of time when skin is continuously exposed to ultrasound. It should be noted that the ultrasound applied should minimally damage the normal cells surrounding the skin cancer, such that less than 10%, less than 5% or less than 1% of the normal cells surrounding the skin cancer are damaged.

A physician can determine the duration of exposure based on, e.g., severity of the disease and other factors. The duration of exposure is no more than 150 minutes, 120 minutes, 90 minutes, 70 minutes, 60 minutes, 40 minutes, or 20 minutes. A subject may need multiple exposure sessions for the same area of skin, and the duration of exposure can be the same or different for each exposure session. Depending on the regimen, each session can be spaced by seconds, minutes, days, weeks, or even months. Prior to each exposure session, a physician can determine the duration of exposure by methods such as visual examination of the cancer or a biopsy of the cancer. During each exposure session, a physician may shorten or lengthen the duration of exposure depending on the temperature of the skin receiving ultrasound exposure, and/or the physician's assessment on the progress of the treatment. By way of example only, if the maximum temperature of the skin receiving ultrasound exposure exceeds a pre-defined value, the ultrasound exposure is terminated or the ultrasound intensity at the focal zone is reduced in order to avoid skin damage or undue discomfort. The pre-defined value can be between 40° C. and 100° C., 40° C. and 90° C., 40° C. and 80° C., 40° C. and 70° C., or 40° C. and 60° C. In some embodiments, cooling techniques can also be used to avoid or lessen skin damage or undue discomfort. These cooling techniques include, but are not limited to, blowing a stream of cold gas (e.g., air, nitrogen gas) at the treatment site.

The method of treating skin cancer using low intensity ultrasound can further comprise the following steps: (i) imaging the skin cancer; (ii) providing input to a control unit as a function of the imaging, wherein the control unit controls an ultrasound transducer; and (iii) adjusting the ultrasound focus, intensity, spatial distribution, or any combination thereof as a function of the input. As used herein, the term “spatial distribution” refers to the spatial distribution of the ultrasound intensity. In some embodiments, the imaging step is performed by the ultrasound transducer to provide spatial information about the cancer and the surrounding tissues. Well known in the art, an ultrasound transducer can not only generate ultrasound, but also receive ultrasound to generate an image. Therefore, ultrasound is routinely used to visualize soft tissues. The spatial information can be two-dimensional or three-dimensional. In some embodiments, the imaging is performed by a temperature sensor (e.g., thermistor, thermocouple, or an infrared sensor), and the sensor can provide spatial temperature information about the cancer and the surrounding tissues. It should be noted that the imaging step does not necessarily require producing an image.

As the control unit receives the information provided by imaging the skin cancer, the control unit can adjust the ultrasound focus, intensity, spatial distribution, or any combination thereof accordingly for maximal therapeutic effect with minimal side effects. By way of examples only, the control unit can change the depth of focus in order to target the skin cancer at a plurality of depths. The control unit can also scan the ultrasound across the skin cancer in a random or pre-determined pattern.

The ultrasound methods provided herein can be used in combination with one or more other methods to treat skin cancer, including excisional surgery, radiation therapy, chemotherapy, freezing, laser therapy, Mohs surgery, curettage and electrodesiccation, photodynamic therapy, or immunotherapy. In some embodiments, the ultrasound method is performed before the use of one or more other methods to treat skin cancer. In some embodiments, the ultrasound method is performed at the same time as the use of one or more other methods to treat skin cancer. In some embodiments, the ultrasound method is performed after the use of one or more other methods to treat skin cancer. For example, excisional surgery can be performed after the ultrasound methods provided herein.

The treatment efficacy of the methods described herein can be determined by the skilled clinician. A treatment is considered “effective treatment,” as the term is used herein, if any one or all of the signs or symptoms of a condition described herein are altered in a beneficial manner, other clinically accepted symptoms are improved, or even ameliorated, or a desired response is induced e.g., by at least 10% following treatment according to the methods described herein. Treatment efficacy can be determined by measuring the percentage of skin cancer cells being killed, for example, at least 10%, 20%, 30%, 40%, 50%, or 90%. Treatment efficacy can also be measured by a failure of an individual to worsen (e.g., cancer no longer spreading).

In some embodiments, the ultrasound method provided herein is used for cosmetic purposes (e.g., removal of skin moles). In some embodiments, the ultrasound method provided herein is used to treat skin cancer at any stage of development including early stage or life-threatening conditions.

In some embodiments, the subject who needs skin cancer treatment is a human or an animal. Usually the animal is a vertebrate such as, but not limited to a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. Additionally, a subject can be an infant or a child.

It some embodiments, factors such as the gender and age of the subject receiving treatment should be taken into account while determining the ultrasound intensity and duration of exposure, as these factors can play a role in the metabolic activity of cells (e.g., normal cells and cancer cells).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although any known methods, devices, and materials may be used in the practice or testing of the invention, the methods, devices, and materials in this regard are described herein.

Some embodiments of the invention are listed in the following numbered paragraphs:
Paragraph 1. A method of treating skin cancer in a subject comprising exposing an area of the skin containing the cancer to low intensity ultrasound, wherein the ultrasound frequency is in the range of about 27 kHz to about 2.2 MHz, and wherein the ultrasound intensity at the focal zone is in the range of about 0.1 to 5 W/cm².
Paragraph 2. The method of paragraph 1, further comprising
   (i) imaging the skin cancer;
   (ii) providing input to a control unit as a function of the imaging, wherein the control unit controls an ultrasound transducer; and
   (iii) adjusting the ultrasound focus, intensity, spatial distribution, or any combination thereof as a function of the input.
Paragraph 3. The method of paragraph 1 or 2, wherein the ultrasound is focused.
Paragraph 4. The method of any of paragraphs 1 to 3, wherein the ultrasound is continuous.
Paragraph 5. The method of any of paragraphs 1 to 4, wherein the duration of exposure is no more than 60 minutes per exposure session.
Paragraph 6. The method of any of paragraphs 1 to 5, wherein the ultrasound frequency is not 2 MHz.
Paragraph 7. The method of any of paragraphs 1 to 6, wherein the skin cancer comprises basal cell carcinoma, squamous cell carcinoma, melanoma, or any combination thereof.
Paragraph 8. The method of paragraph 7, wherein the skin cancer comprises melanoma.
Paragraph 9. The method of any of paragraphs 1 to 8, wherein the subject is a mammal.
Paragraph 10. The method of paragraph 9, wherein the subject is a human.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean ±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased morbidity or mortality. The term “treatment” of a disease also includes providing relief from the symptoms or side-effects of the disease (including palliative treatment).

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

### In vitro Studies of Cellular Response to Low Intensity Ultrasound

Method and materials. The A375s were grown in Dulbecco's Modified Eagle Medium (DMEM) supplemented with 10% fetal bovine serum (FBS). The HEKas were grown in EpiLife media supplemented with keratinocyte growth factor. To prepare for sonication, cells were first grown to 80-90% confluency in flasks incubated at 37° C. and 5% CO₂ before being trypsinized using trypsin with EDTA and plated in a gradually increasing gradient of cell density in 96-well plates. Column 1 was a blank, media sans cells, and column 12 of each plate was also held as a blank with autoclaved double distilled water because it was a relative “cold” spot in terms of ultrasonic power density given the instrumental setup used (described in more detail below). Cell density in the remaining columns were as follows:
Column 2: 1×10⁴ cells/mL; Column 3: 2×10⁴ cells/mL; Column 4: 3×10⁴ cells/mL; Column 5: 4×10⁴ cells/mL; Column 6: 5×10⁴ cells/mL; Column 7: 6×10⁴ cells/mL; Column 8: 8×10⁴ cells/mL; Column 9: 1×10⁵ cells/mL; Column 10: 1.2×10⁵ cells/mL; Column 11: 1.6×10⁵ cells/mL.

Each concentration was plated in all eight wells in each column. The plates were put into the incubator overnight. The following day, they were treated. The experimental treatment setup consisted of a sonicator tank full of water. The plate was suspended over the water such that the bottom surface made contact with the surface of the water. Treatment involved sonication at 27 kHz with an energy density of 0.17 W/cm² for 2 minutes, as calibrated on plates with only double distilled water using a bolometry technique described below. Immediately following treatment, the plates were visually inspected and returned to the incubator overnight. The untreated control plates stayed out of the incubator in the hood while the others were being treated for the sake of continuity, and everything was returned to the hood simultaneously. The next day, overall metabolic activity was assessed via WST-1 assay (manufactured by Roche). The WST-1 assay measures metabolic activity by cellular dehydrogenase activity. In the presence of active dehydrogenases, WST-1 will change its fluorescence. The delta value between the unchanged and changed WST-1 reflects the level of cellular activity.

The equipment included a 27 kHz transducer with which to sonicate cells. Given the frequency, the wavelength was calculated and ˜3 full wavelengths were allowed to propagate through the conductive media (degassed water) before reaching the plate and the cells to insure a more uniform energy distribution. This calculation informed the development of the rig that held each plate. Energy density was calculated with thermocouples that assessed temperature change in select wells throughout the entire plate. Each well held 200 uL of media. In the calibrations, distilled water was used . Based upon the slope of the temperature change, the total energy deposited in each well can be calculated. The energy density averaged 0.17+/−0.03 W/cm² across the wells tested.

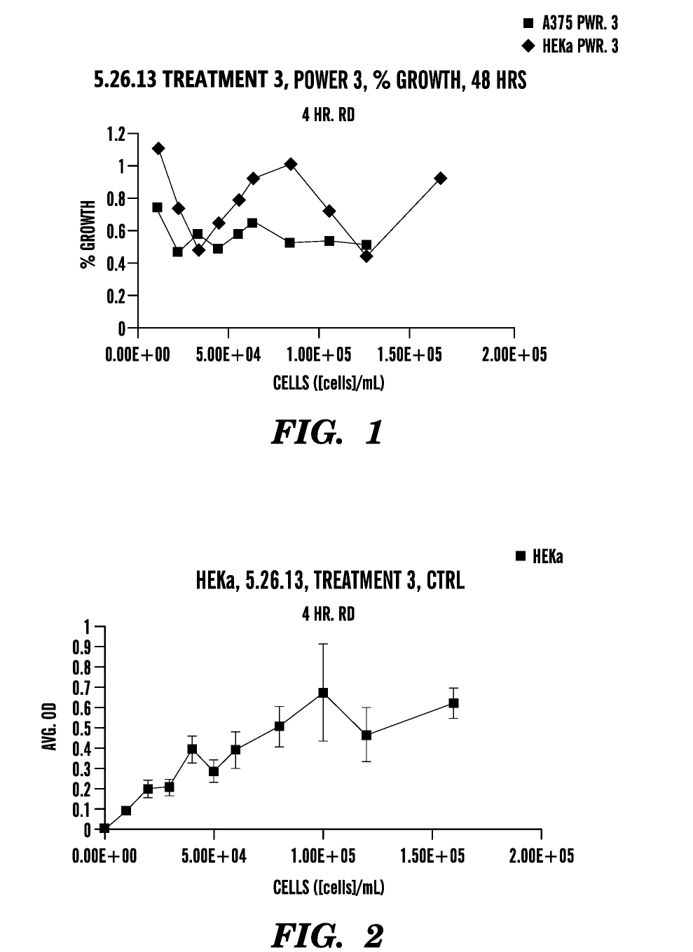

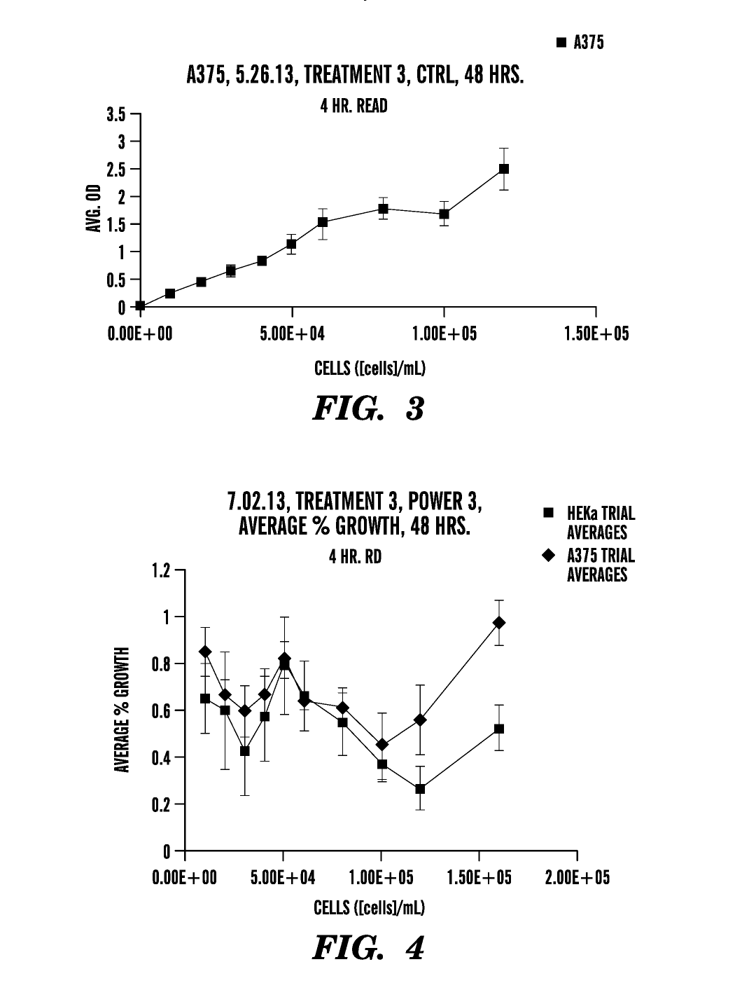

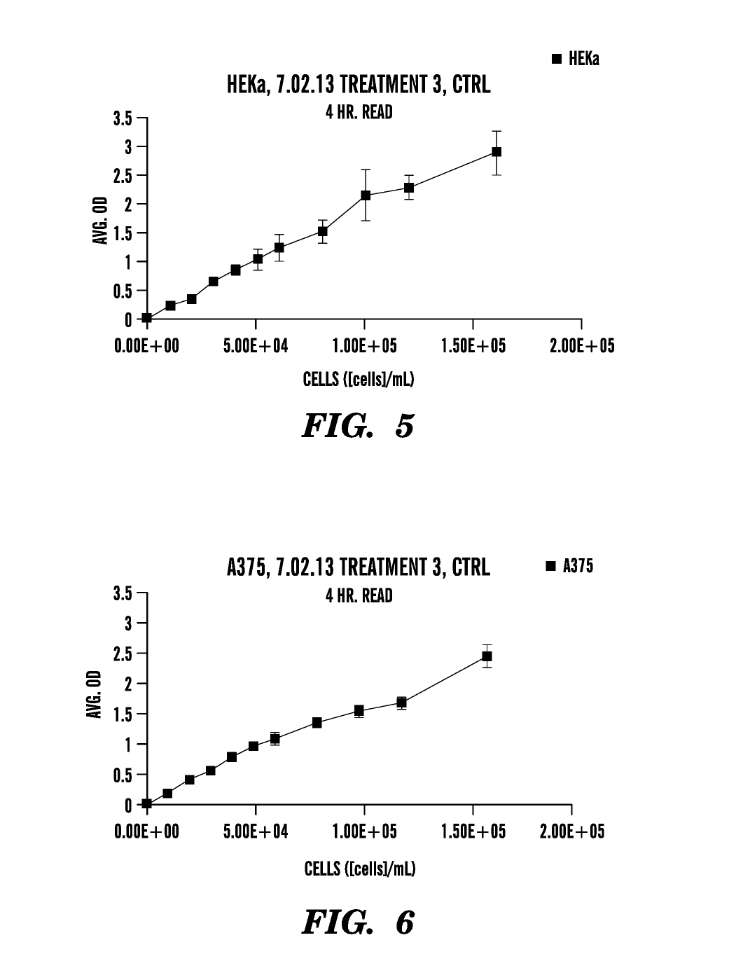

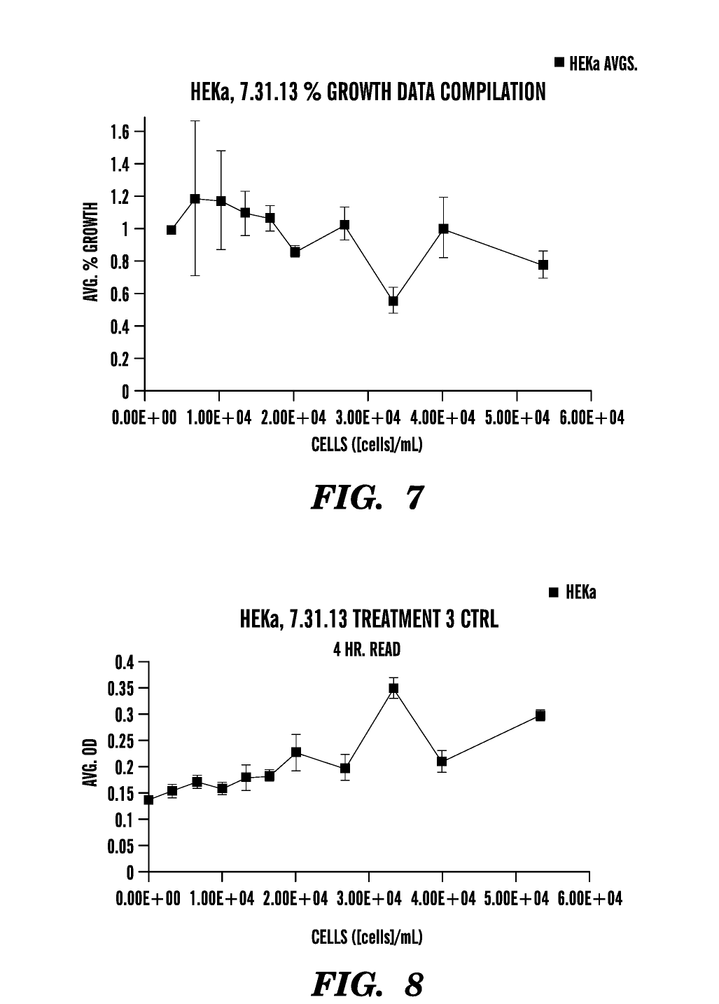

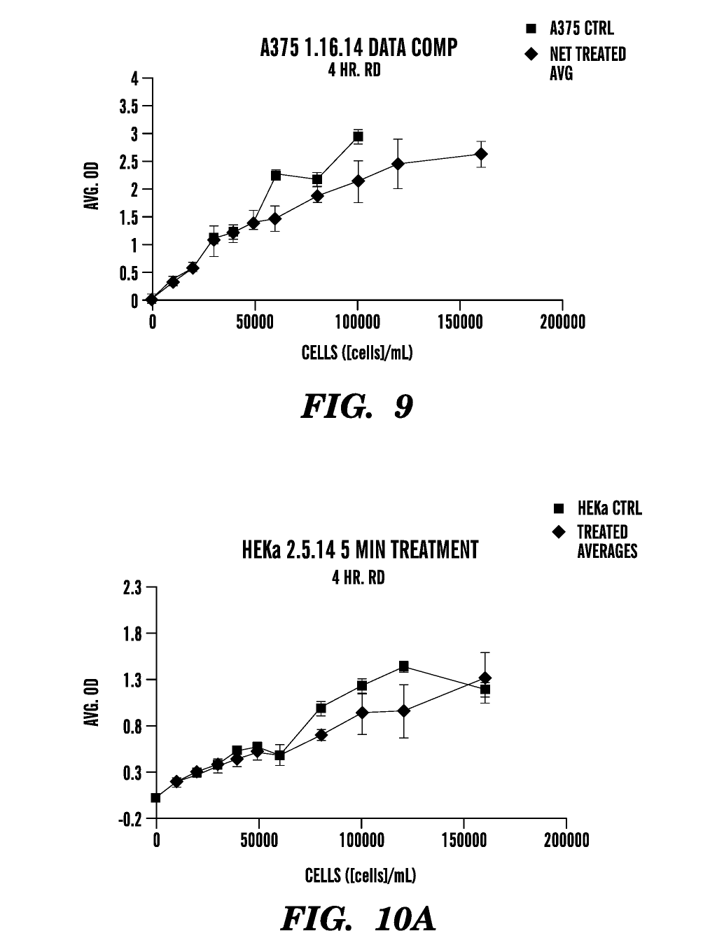

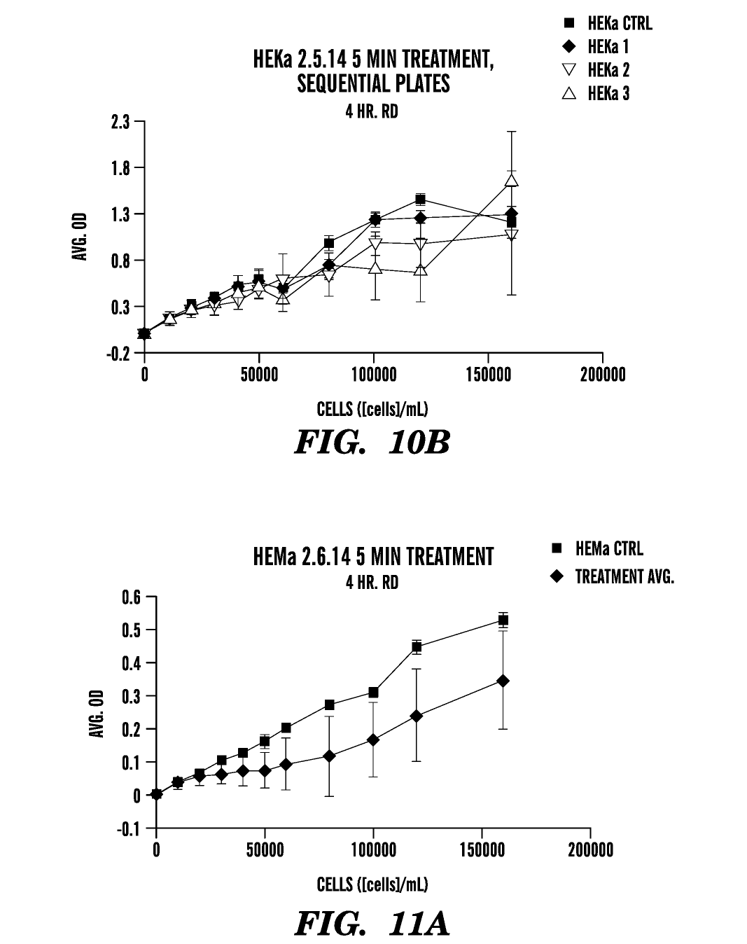

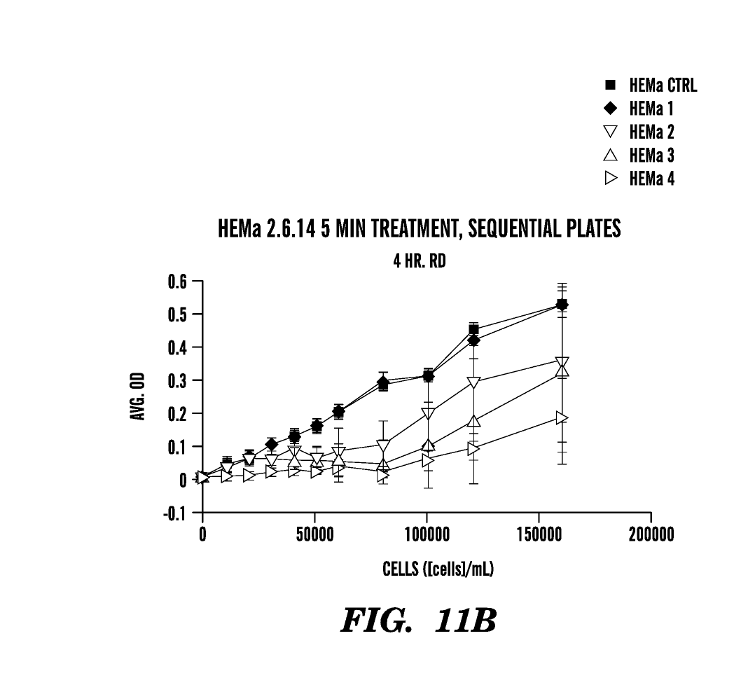

Results. The data revealed selective survival based on metabolic activity. FIG. 1 shows the percent growth following treatment of both A375 and HEKa, and HEKa appears to survive better than A375. The metabolic activity of the untreated HEKa control (FIG. 2) and A375 control (FIG. 3) was used to calculate percent growth shown in FIG. 1. FIG. 4 shows the percent growth following treatment of both A375 and HEKa in a second trial. The metabolic activity of the untreated HEKa control (FIG. 5) and A375 control (FIG. 6) was used to calculate percent growth shown in FIG. 4. A375s were consistently observed to suffer following the ultrasound treatment. HEKas were not as clear-cut. They have a shorter period of viable culture time. When a culture is started fresh from cryo storage, they reproduce rapidly and show robust growth. As they age in culture, they gradually slow down, reproducing less frequently until they eventually stop altogether. HEKas nearing the end of their reproductive time in culture show greater survival (FIGS. 7 & 8) following ultrasound treatment compared to the robustly metabolizing newer cultures. Increased metabolic levels and more robust mitotic activity made both cancerous A375s and noncancerous HEKas susceptible to ultrasound damage. Visual inspection immediately post-treatment showed that there were fewer cells in the wells of the affected plates.

### Example 2

### In vitro Studies of Cellular Response to Temperature Increase Resulting from Low Intensity Ultrasound

Within the megahertz range, low intensity ultrasound affects the growth and proliferation of melanoma in vitro. Two noncancerous cell lines, adult human epidermal melanocytes (HEMa) and adult human epidermal keratinocytes (HEKa) were tested as controls in addition to the A375 melanoma line. Frequency of the transducer used was 2.2 MHz at an intensity of 0.5 W/cm²±0.04. HEMa and HEKa cells exhibited a temperature dependence; their growth was unaffected by the treatment initially, but as the transduction medium, the water bath, heated up with subsequent treatments, survival of the two control cell lines became more erratic. In contrast, the A375 line showed statistically significant consistent decreased growth following treatment sans temperature dependence.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1. A method of treating skin cancer in a subject comprising exposing an area of the skin containing the cancer to low intensity ultrasound, wherein the ultrasound frequency is in the range of about 27 kHz to about 2.2 MHz, and wherein the ultrasound intensity at the focal zone is in the range of about 0.1 to 5 W/cm².

2. The method of claim 1, further comprising
(i) imaging the skin cancer;
(ii) providing input to a control unit as a function of the imaging, wherein the control unit controls an ultrasound transducer; and
(iii) adjusting the ultrasound focus, intensity, spatial distribution, or any combination thereof as a function of the input.

3. The method of claim 1, wherein the ultrasound is focused.

4. The method of claim 1, wherein the ultrasound is continuous.

5. The method of claim 1, wherein the duration of exposure is no more than 60 minutes per exposure session.

6. The method of claim 1, wherein the ultrasound frequency is not 2 MHz.

7. The method of claim 1, wherein the skin cancer comprises basal cell carcinoma, squamous cell carcinoma, melanoma, or any combination thereof.

8. The method of claim 7, wherein the skin cancer comprises melanoma.

9. The method of claim 1, wherein the subject is a mammal.

10. The method of claim 9, wherein the subject is a human.

What is claimed is:

1. A method of treating skin cancer in a subject comprising exposing an area of the skin containing the cancer to low intensity ultrasound, wherein the ultrasound frequency is in the range of about 27 kHz to about 2.2 MHz, and wherein the ultrasound intensity at the focal zone is in the range of about 0.1 to 5 W/cm².

2. The method of claim 1, further comprising
(i) imaging the skin cancer;
(ii) providing input to a control unit as a function of the imaging, wherein the control unit controls an ultrasound transducer; and
(iii) adjusting the ultrasound focus, intensity, spatial distribution, or any combination thereof as a function of the input.

3. The method of claim 1 or 2, wherein the ultrasound is focused.

4. The method of any of claims 1 to 3, wherein the ultrasound is continuous.

5. The method of any of claims 1 to 4, wherein the duration of exposure is no more than 60 minutes per exposure session.

6. The method of any of claims 1 to 5, wherein the ultrasound frequency is not 2 MHz.

7. The method of any of claims 1 to 6, wherein the skin cancer comprises basal cell carcinoma, squamous cell carcinoma, melanoma, or any combination thereof.

8. The method of claim 7, wherein the skin cancer comprises melanoma.

9. The method of any of claims 1 to 8, wherein the subject is a mammal.

10. The method of claim 9, wherein the subject is a human.
